(19) United States
(12) Patent Application Publication
(10) Pub. No.: US 2018/0099135 A1
(43) Pub. Date: Apr. 12, 2018

(54) MEDICAL FLUID LINE CONNECTOR

(71) Applicant: Fresenius Medical Care Holdings, Inc., Waltham, MA (US)

(72) Inventors: Devon Hall, Clinton, UT (US); Michael Parry Smith, Ogden, UT (US)

(21) Appl. No.: 15/718,060

(22) Filed: Sep. 28, 2017

Related U.S. Application Data
(60) Provisional application No. 62/407,308, filed on Oct. 12, 2016.

(51) Int. Cl. A61M 39/10; A61M 1/28; A61J 1/10

(52) U.S. Cl. CPC A61M 39/10; A61M 2039/1061; A61J 1/10; A61M 1/285

(57) ABSTRACT

A medical fluid line connector includes a main body configured to accept a corresponding connector, where the main body defines an opening permitting fluid communication between an interior region of the medical fluid line connector and the corresponding connector when the corresponding connector is connected to the main body. The connector also includes a breakaway body detachably connected to the main body, such that, when the breakaway body is inserted into an open end of a flexible medical fluid line, the breakaway body has a portion creating an interference fit against an inner surface of the flexible medical fluid line, and the breakaway body is detachable from the maim body such that detachment of the breakaway forms a second opening in the main body that permits fluid communication between the medical fluid line and the interior region of the medical fluid line connector.

### CROSS REFERENCE OF RELATED APPLICATION

This application claims priority to U.S. Provisional Application No. 62/407,308 filed Oct. 12, 2016, which is incorporated herein by reference.

### TECHNICAL FIELD

This disclosure relates to flexible medical fluid tubing connectors for medical fluid pumping systems and related devices and methods.

### BACKGROUND

Dialysis is a treatment used to support a patient with insufficient renal function. The two principal dialysis methods are hemodialysis and peritoneal dialysis.

During hemodialysis (“HD”), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. During peritoneal dialysis (“PD”), a patient's peritoneal cavity is periodically infused with dialysis solution or dialysate. The membranous lining of the patient's peritoneum acts as a natural semi-permeable membrane that allows diffusion and osmosis exchanges to take place between the solution and the blood stream.

In either form of dialysis, often a large quantity of dialysis solution or dialysate is pumped by a dialysis machine, either into a dialyzer (HD) or into the patient (PD). The dialysis solution is typically stored in medical fluid bags prior to delivery of the dialysis solution to the dialysis device. During a typical dialysis procedure, one or more medical fluid containers of dialysis solution are connected to the dialysis machine for use in the dialysis process and to facilitate ease of use of the medical fluid bags in the dialysis procedure, the medical fluid bags are equipped with a medical fluid line connector to establish a fluid connection to a delivery line of the dialysis machine.

### SUMMARY

Certain aspects of the present invention relate to a medical fluid line connector including a main body configured to accept a corresponding connector, where the main body defines a first opening that permits fluid communication between an interior region of the medical fluid line connector and the corresponding connector when the corresponding connector is connected to the main body. The medical fluid line connector also includes a breakaway body detachably connected to the main body, with the breakaway body configured to be inserted into an open end of a flexible medical fluid line and the breakaway body having a portion configured to create an interference fit against an inner surface of the flexible medical fluid line when the breakaway body is inserted into the flexible medical fluid line. The breakaway body and the main body are configured such that detachment of the breakaway body from the main body forms a second opening in the main body that permits fluid communication between the medical fluid line and the interior region of the medical fluid line connector.

In some examples, the breakaway body at least partially defines a fluid passageway configured to permit fluid flowing through the medical fluid line to flow past the breakaway body and into the second opening.

In some examples, an intersection of the main body and the breakaway body defines a breakaway region configured to enable a force applied to the breakaway body to separate the breakaway body from the main body at the breakaway region.

In some examples, the breakaway region includes a thin wall region. In some examples, the breakaway region includes a frangible region.

In some examples, the medical fluid line connector includes an exterior sleeve around a periphery of the main body, where a portion of the exterior sleeve is radially spaced from the main body and creates a sleeve cavity configured to receive an open end of medical fluid line and seal the open end of medical fluid line around an exterior surface of the main body.

In some examples, the breakaway body includes a plurality of protrusions, and a portion of a terminal edge of each protrusion is sized and positioned to create the interference fit against the inner surface of the flexible tube.

In some examples, the terminal edges of the plurality of protrusions define an arcuate shape. In some examples, the plurality of protrusions together form a substantially spherical shape.

In some examples, the plurality of protrusions define a corresponding plurality of gaps between adjacent protrusions, and the gaps and the inner surface of the flexible tube together define a plurality of fluid passageways configured to permit fluid flowing through the medical fluid line to flow past the breakaway body and into the second opening.

In some examples, the radial protrusions include longitudinal ribs. In some examples, the longitudinal ribs span the length of the breakaway body.

In some examples, the connector further includes a connector interface body attached to the main body, the connector interface body includes interface features configured to mate with corresponding interface features on the corresponding connector and securely attached the corresponding connector to the medical fluid line.

In some examples, the flexible medical fluid line is coupled to a container containing fluid for use in a dialysis operation, and the connector is configured to seal the bag by sealing the open end of the flexible medical fluid line.

In some examples, the corresponding connector is a corresponding connector of a medical fluid line of a dialysis system.

In some examples, the medical fluid line extends from a medical fluid container. In some examples, the medical fluid container is a dialysis solution container. In some examples, the medical fluid line extends from a medical fluid bag. In some examples, the medical fluid bag is a dialysis solution bag.

Another example is a system that includes a medical fluid container, a flexible medical fluid line connected to the medical fluid container, and a medical fluid line connector connected to the medical fluid line. The medical fluid line connector includes a main body configured to accept a corresponding connector, and the main body defines a first opening to permit fluid communication between an interior region of the medical fluid line connector and the corresponding connector when the corresponding connector is connected to the main body. The medical fluid line connector also includes a breakaway body detachably connected to the main body, with the breakaway body disposed inside of an open end of the flexible medical fluid line, and the breakaway body having a portion that defines an interference fit against an inner surface of the flexible medical fluid line. The breakaway body and the main body are configured such that detachment of the breakaway body from the main body forms a second opening in the main body that permits fluid communication between the medical fluid container and the interior region of the medical fluid line connector.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a peritoneal dialysis (“PD”) system that includes a PD cycler positioned atop a portable cart from which multiple medical fluid bags hang.

FIG. 2 is a perspective view of the PD cycler and a PD cassette of the PD system of FIG. 1. A door of the PD cycler is in the open position to show the inner surfaces of the PD cycler that interface with the PD cassette during use.

FIG. 3 is a cross-section illustration of a medical fluid line connector attached to a flexible medical fluid line of one of the medical fluid bags illustrated in FIG. 1, with a breakaway body of the connector disposed in the flexible medical fluid line.

FIG. 4 is a cross-section illustration of the medical fluid line connector, flexible medical fluid line, and medical fluid bag assembly of FIG. 3, showing a corresponding connector attached to the medical fluid line connector and the breakaway body detached from a main body of the medical fluid line connector.

FIG. 5 is a perspective illustration of the medical fluid line connector that is shown in FIGS. 3 and 4.

FIGS. 6A and 6B are side and cross-sectional views, respectively, of the medical fluid line connector of FIG. 5.

FIGS. 7A-7D are cross-section illustrations of the medical fluid line connector being attached to the flexible medical fluid line of the medical fluid bag and subsequent attachment of the corresponding connector and detachment of the breakaway body from the main body of the medical fluid line connector.

FIG. 8 is a perspective view of the breakaway body attached to the main body of the medical fluid line connector of FIG. 5.

FIGS. 9A and 9B are cross-sectional and exploded views of interface components attached to the main body of the medical fluid line connector of FIG. 5.

FIGS. 10A and 10B are cross-sectional and perspective illustrations, respectively, of the corresponding connector coupled to the interface components of the medical fluid line connector of FIGS. 9A and 9B.

FIG. 11 is a perspective illustration of a computational simulation of the deflection of the breakaway body of the medical fluid line connector of FIG. 5.

FIG. 12 is a perspective illustration of an alternative design of a medical fluid line connector.

FIG. 13 is a perspective illustration of an alternative design of a medical fluid line connector.

### DETAILED DESCRIPTION

This disclosure relates generally to medical fluid line connectors that may, in some instances, be used with medical fluid pumping systems and related devices and methods. In some cases, a medical fluid pumping system (e.g., a peritoneal dialysis (“PD”) system) includes a medical fluid pumping machine (e.g., a PD cycler) having a piston with a piston head that can be mechanically connected to a medical fluid cassette (e.g., a PD fluid cassette). Typically, the cassette includes a flexible membrane and a fastening member (e.g., a dome-shaped fastening member) attached to the membrane. The membrane and the fastening member overlie a recessed region of a rigid base of the cassette to form a fluid pump chamber, and the piston of the medical fluid pumping machine is designed to be mechanically connected to the fastening member of the cassette. With the piston of the medical fluid pumping machine mechanically connected to the fastening member of the cassette, reciprocation of the piston causes fluid to be alternately drawn into (and then forced out) the fluid pump chamber from a bag containing dialysate solution by pulling the fastening member and membrane away from the recessed region of the base and then advancing the fastening member and membrane toward the recessed region of the base.

In peritoneal dialysis, a bag containing dialysate solution is typically fluidly connected to the PD fluid cassette using a connector to maintain a sterile barrier after connection with external tubing. After being connected to tubing, a breakaway piece of the connector disposed in the external tubing is broken and translated away from the connector. The disconnection of the breakaway piece from the connector opens and maintains the fluid pathway out of the bag, allowing solution to leave the bag and enter the patient's peritoneum. Aspects of the present medical fluid line connector include a breakaway piece designed to create an interference fit with the tubing to maintain the gap after breaking the breakaway piece to keep consistent flow during treatment. In this way, the breakaway piece can be held spaced apart from the connector body without maintaining any connection to the connector body.

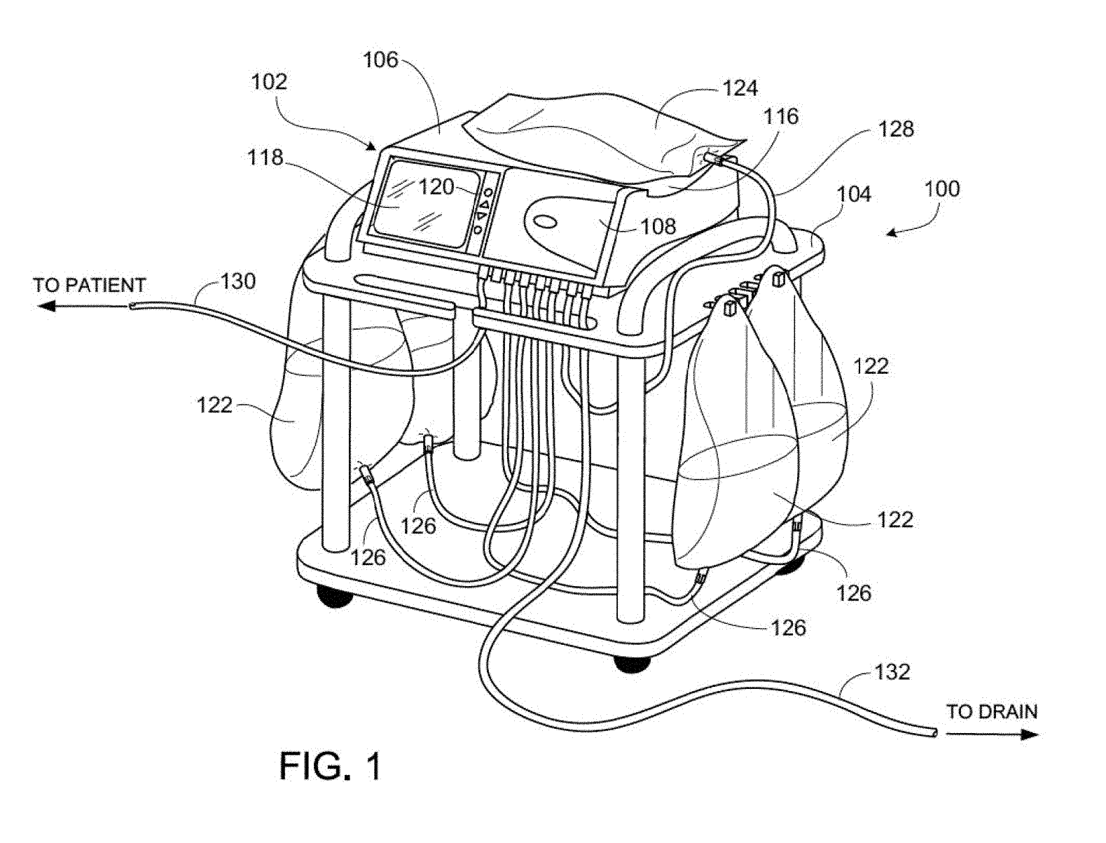

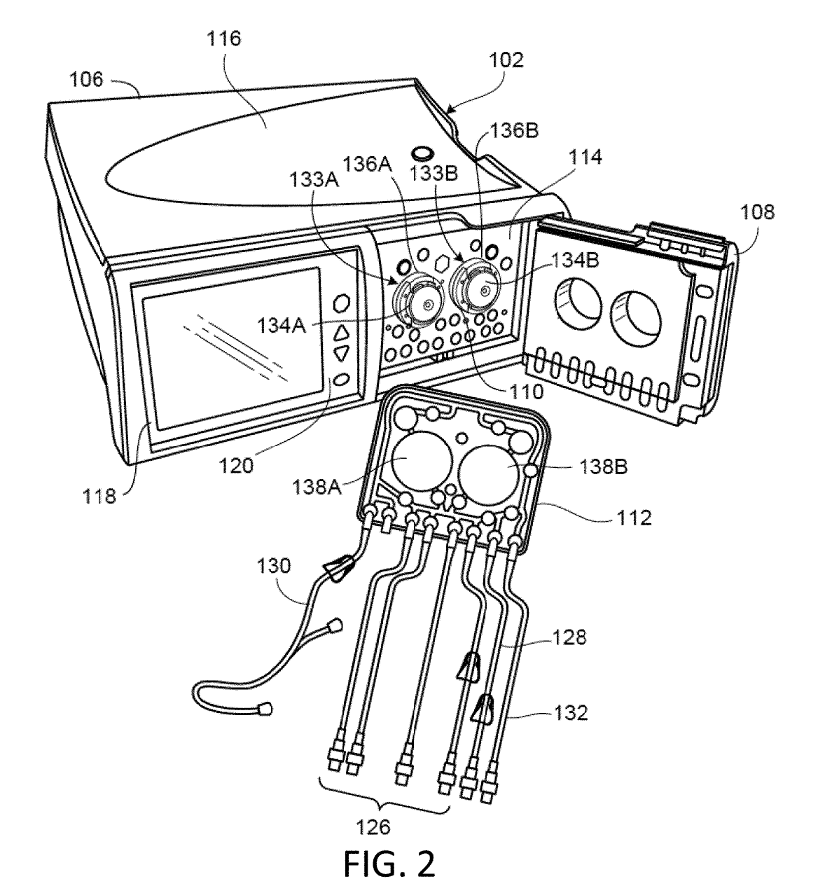

Referring to FIG. 1, a PD system 100 includes a PD cycler (also referred to as a PD machine) 102 seated on a cart 104. Referring also to FIG. 2, the PD cycler 102 includes a housing 106, a door 108, and a cassette interface 110 that abuts a disposable PD cassette 112 when the cassette 112 is disposed within a cassette compartment 114 formed between the cassette interface 110 and the closed door 108. A heater tray 116 is positioned on top of the housing 106. The heater tray 116 is sized and shaped to accommodate a bag of dialysis solution (e.g., a 5-liter bag of dialysis solution). The PD cycler 102 also includes a touch screen 118 and additional control buttons 120 that can be operated by a user (e.g., a patient) to allow, for example, set-up, initiation, and/or termination of a PD treatment.

Dialysis solution bags 122 are suspended from fingers on the sides of the cart 104, and a heater bag 124 is positioned on the heater tray 116. The dialysis solution bags 122 and the heater bag 124 are connected to the cassette 112 via dialysis solution bag lines 126 and a heater bag line 128, respectively. The dialysis solution bag lines 126 can be used to pass dialysis solution from dialysis solution bags 122 to the cassette 112 during use, and the heater bag line 128 can be used to pass dialysis solution back and forth between the cassette 112 and the heater bag 124 during use. In addition, a patient line 130 and a drain line 132 are connected to the cassette 112. The patient line 130 can be connected to a patient's abdomen via a catheter and can be used to pass dialysis solution back and forth between the cassette 112 and the patient during use. The drain line 132 can be connected to a drain or drain receptacle and can be used to pass dialysis solution from the cassette 112 to the drain or drain receptacle during use.

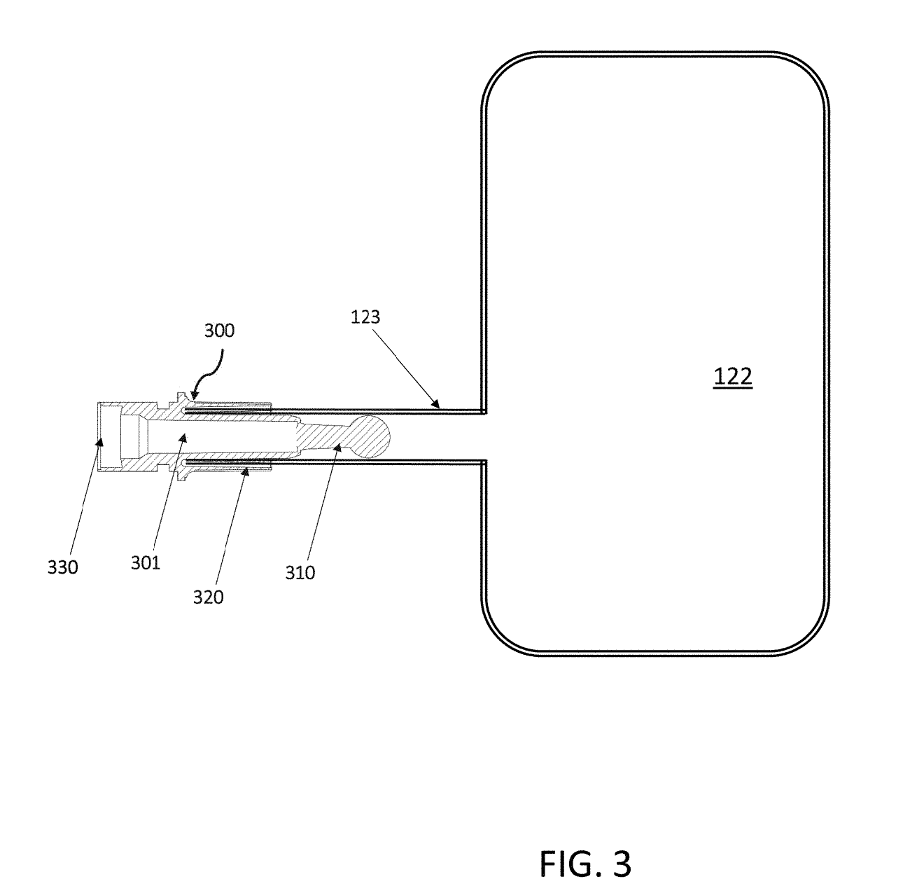

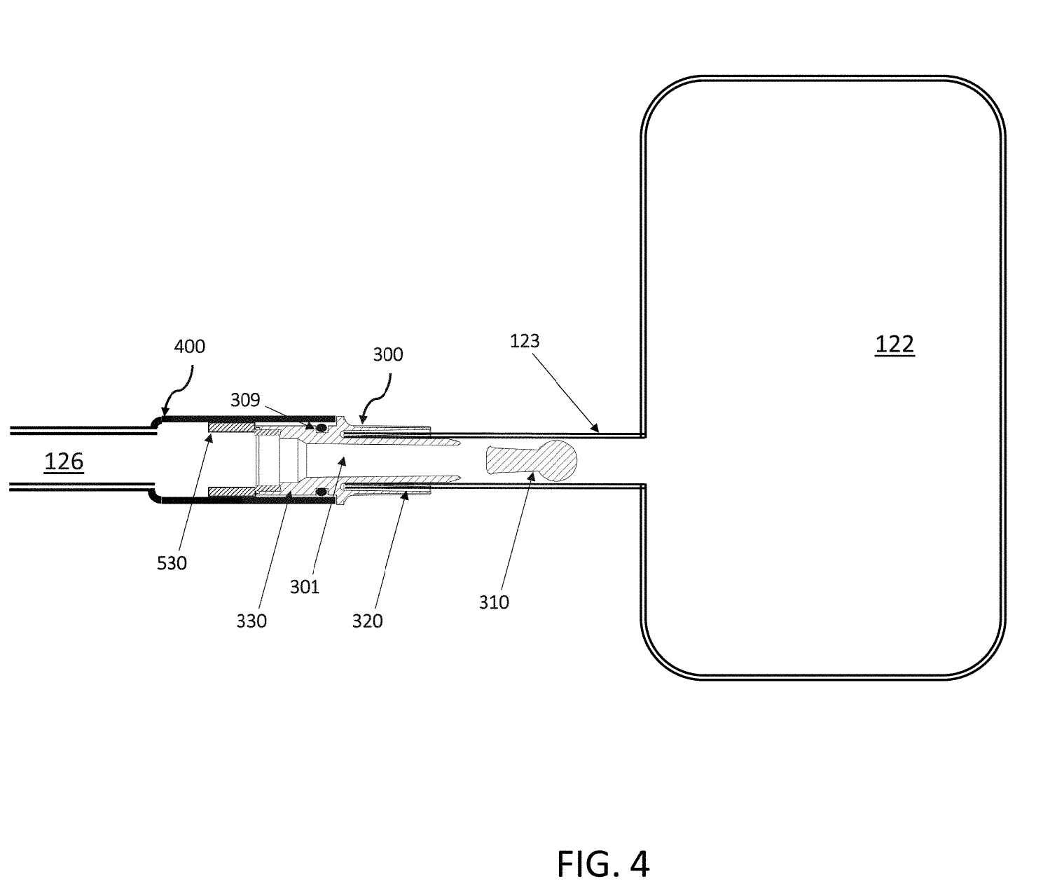

FIG. 3 is a cross-section illustration of a medical fluid line connector 300 attached to a flexible medical fluid line 123 of the medical fluid bag 122, with a breakaway body 310 of the medical fluid line connector 300 disposed in flexible medical fluid line 123. An open end of the flexible medical fluid line 123 is inserted in a peripheral sleeve 320 of the medical fluid line connector 300, and the peripheral sleeve 320 seals the open end of the medical fluid line 123 around the medical fluid line connector 300 such that the medical fluid line connector 300 forms a sealed plug in the flexible medical fluid line 123. The medical fluid line connector 300 includes a tubular main body forming an inner fluid cavity 301 with an open end 330 into the inner fluid cavity 301. An intersection between the tubular main body of the medical fluid line connector 300 and the breakaway body 310 forms the barrier between the inner fluid cavity 301 of the medical fluid line connector 300 and the flexible medical tubing 123. In the configuration shown in FIG. 3, the medical fluid bag 122 is sealed by the medical fluid line connector 300 and may contain a medical fluid (not shown). In this configuration, the medical fluid bag 122, along with the sealed flexible medical fluid line 123 is ready to supply a contained medical fluid to, for example, a medical fluid device after attaching a corresponding connector to the medical fluid line connector 300, as shown in FIG. 4, or may be stored for later use or transported to a treatment location.

FIG. 4 is a cross-section illustration of the medical fluid line connector 300, flexible medical fluid line 123, and medical fluid bag 122 assembly of FIG. 3, showing a corresponding connector 400 attached to the medical fluid line connector 300 and showing the breakaway body 310 detached from the medical fluid line connector 300 to permit fluid communication between the medical fluid bag 122 and the interior of the main body of the medical fluid line connector 300. The detachment of the breakaway body 310 from the medical fluid line connector 300 opens and maintains a fluid pathway out of the medical fluid bag 122, allowing a fluid or solution to leave the medical fluid bag 122, pass into the inner fluid cavity 310 of the medical fluid line connector 300, and pass into the dialysis solution bag line 126 connected to, for example, the PD cycler 102. The corresponding connector 400 is coupled to the medical fluid line connector 300 by way of an interface component 530 attached to the medical fluid line connector 300. The interface component 530 is configured to removeably and securely engage the corresponding connector 400 to the medical fluid line connector 300 using, for example, exterior threads engaged with corresponding interior threads on the corresponding connector 400. The medical fluid line connector 300 also includes an O-ring seal 309 that interfaces with the corresponding connector 400 to ensure a leak-free fluid connection between the dialysis solution bag line 126 and the inner fluid cavity 301 of the medical fluid line connector 300.

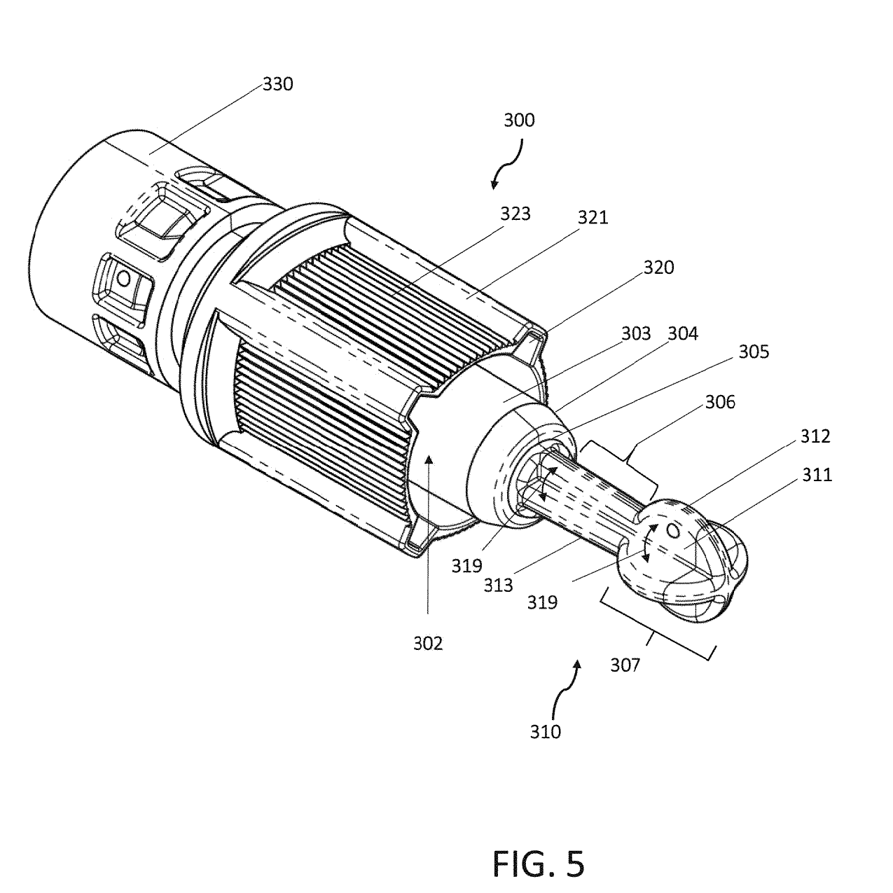

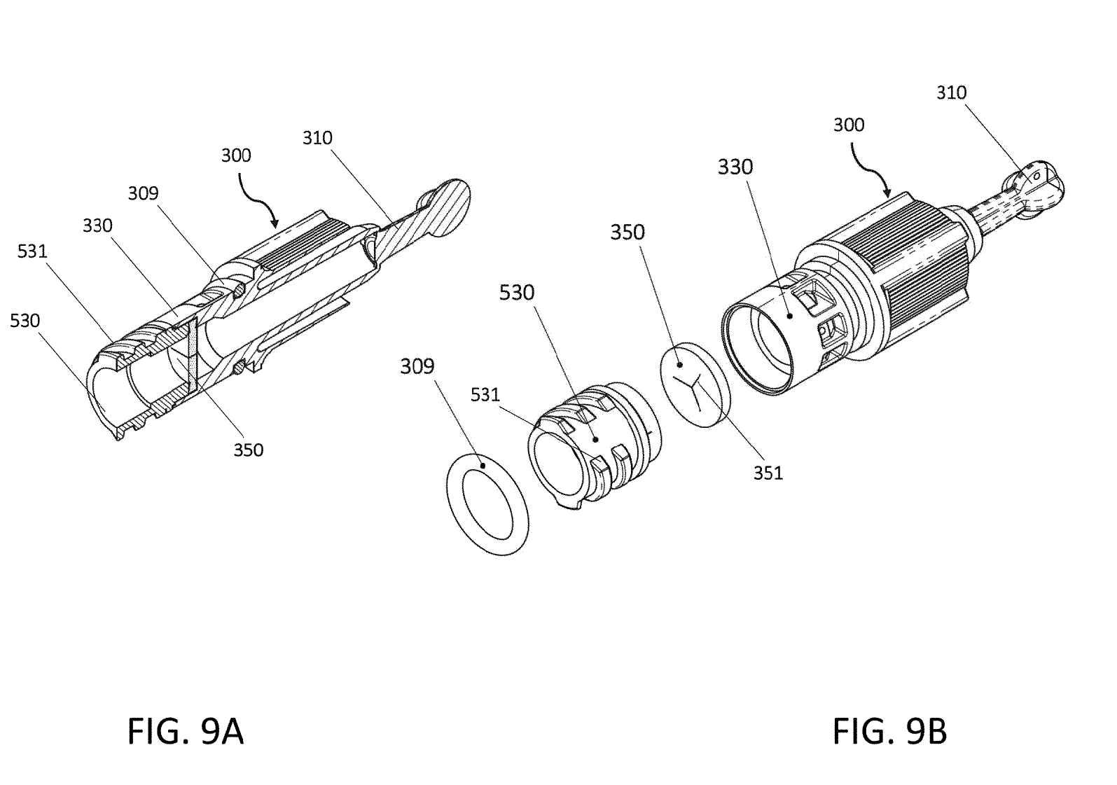

FIG. 5 is a perspective illustration of the medical fluid line connector 300. The medical fluid line connector 300 includes a proximal open end 330, a peripheral exterior sleeve 320 around a main body 303, and a breakaway body 310 that forms a closed distal end of the medical fluid line connector 300. The proximal open end 330 is configured to attach to an interface body (show in FIGS. 9A and 9B). The peripheral exterior sleeve 320 forms a sleeve cavity 302 around a main body 302 and the sleeve cavity 302 is sized and shaped to accept the open end of a flexible medical fluid line 123. The exterior surface of the peripheral exterior sleeve 320 is adapted to be gripped by a user's hand or a tool, in order to assist in coupling the corresponding connector 400 to the medical fluid line connector 300 and assist in breaking the breakaway body 310 from the main body 303. The peripheral exterior sleeve 320 may also be described as a skirt because one end of the peripheral exterior sleeve 320 is incorporated with the main body 303 of the medical fluid line connector 300 and the opposite end defines the sleeve cavity 302. To aid hand-gripping of the medical fluid line connector 300, the peripheral exterior sleeve 320 includes a gripping surface 323 formed into the exterior surface of exterior sleeve 320. Additionally, the exterior sleeve 320 includes four tool protrusions 321 at four corner to provide parallel sections of a wrench to engage and apply torque to the exterior sleeve 320. The breakaway body 310 intersects the main body 303 at a breakaway region 305, which may be, for example, a thin-walled region or a frangible region configured to enable the breakaway body 310 to be separated from the main body 303 when a force is applied to the breakaway body 310 sufficient to cause material separation at the breakaway region.

The breakaway body 310 includes a tip region 307 at the opposite end from the breakaway region 305 configured to be inserted into an open end of a flexible medical fluid line 123. The breakaway body 310 includes a plurality of longitudinal ribs 311 along the length of the breakaway body 310. The longitudinal ribs 311 define straight tapered radial edges 313 spanning the breakaway body 310 from the tip region 307 to the breakaway region 305. The four longitudinal ribs 311 are arranged a cross-configuration along the longitudinal axis of the breakaway body 310, and voids 319 between adjacent longitudinal ribs 311 define fluid passageways long the breakaway body 310 when the breakaway body 310 is inserted into the flexible medical fluid line 123. The longitudinal ribs 311 at the tip region 307 of the breakaway body 310 have arcuate radial edges 312 that form a “ball” at the tip region 307 of the breakaway body 310. The arcuate radial edges 312 guide the insertion of the breakaway body 310 in the flexible medical fluid line 123 and are rounded to prevent of structural damage to the flexible medical fluid line 123 during insertion and during deflection of the breakaway body 310 from the main body 303, as a deflection operation may include pressing on the flexible medical fluid line 123 to apply a sufficient force on the breakaway body 310 to detach the breakaway body 310 from the main body 303. The longitudinal ribs 311 extend along the length of the breakaway body 310, and in a base region 306 the longitudinal ribs 311 have tapering radial edges 313 that define a conic exterior profile from the breakaway region 305 to the arcuate edges 312 the tip region 307. The diameter of the longitudinal ribs 311 in the base region 306 is smaller than the diameter of the inner surface of the flexible medical fluid line 123. Therefore, the longitudinal ribs 311 in the base region 306 expand the effective cross-sectional area of the flow passages created by the voids 319 beyond the “ball” at the tip region 307 and reduce the overall flow restriction caused by the breakaway body 310. Additionally, a width of the tapering radial edges 313 of the breakaway body 310 at in the base region 306 includes a diameter of the base breakaway region 305 being less than an inner diameter the flexible medical line 123, which is required for fluid flow between the breakaway region 305 and the flexible medical line 123. Accordingly, the main body 303, which has an exterior surface sized to accept the inner surface flexible medical line 123, includes a tapered region 304 reducing the diameter of the main body 303 adjacent to the breakaway region 304. In this manner, separation of the breakaway body 310 from the main body 303 permits fluid to pass around the breakaway region 305.

In some embodiments, the medical fluid line connector 300 includes features that improve molding quality of the part. For example, the walls of the medical fluid line connector 300 can be designed to have uniform thickness to reduce quality risk by reducing shrinkage and void affects when wall or area of the medical fluid line connector 300 are too thick. The longitudinal ribs 311 of the breakaway body 310 are tapered at their radial edges in the base region 306, but may define a constant thickness at their root to reduce the overall thickness of the breakaway body 310.

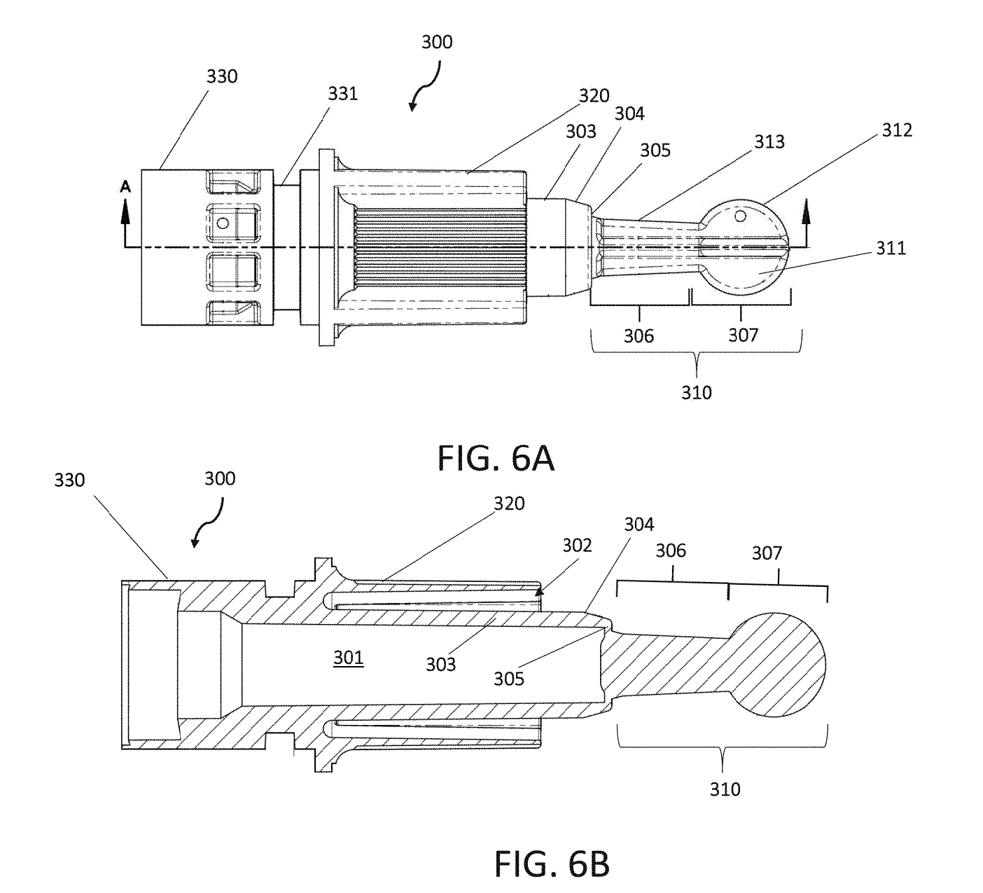

FIGS. 6A and 6B are side and cross-sectional views, respectively, of the medical fluid line connector 300 of FIG. 5. FIG. 6A shows the side view of FIG. 5 and shows an indentation in the medical fluid line connector 300 configured to receive and secure the O-ring 309 to seal the corresponding connector 400 when it is placed around the open end 330. The conic profile of the base region 306 of the breakaway body is visible, along with the spherical profile of the tip region 307. FIG. 6B is a cross-section view of FIG. 6A, taken along datum “A.” FIG. 6B shows the inner fluid cavity 301 of the medical fluid line connector 300 formed by the main body 303, with the inner fluid cavity 301 extending from the open end 330 to the base region 306 of the breakaway body 310. The breakaway region 305 is located at the intersection of tapered region 304 of the main body 303 and the base region 306 of the breakaway body 310. The breakaway region 305 is shown as a thin-walled region, which is, in some instances, a frangible region or similarly destructible region configured to enable forced detachment of the breakaway body 310 from the main body 303. Also shown is the sleeve cavity 302 extending between the peripheral sleeve 320 and the main body 303. The sleeve cavity 302 is configured to secure the open end of the flexible medical fluid line 123 to the medical fluid line connector 300, and may, in some instances, be configured to contain an adhesive or bonding adjacent to facility a secure bond of the flexible medical fluid line 123 an exterior surface of the main body 303 and/or to an inner surface of the peripheral sleeve 320.

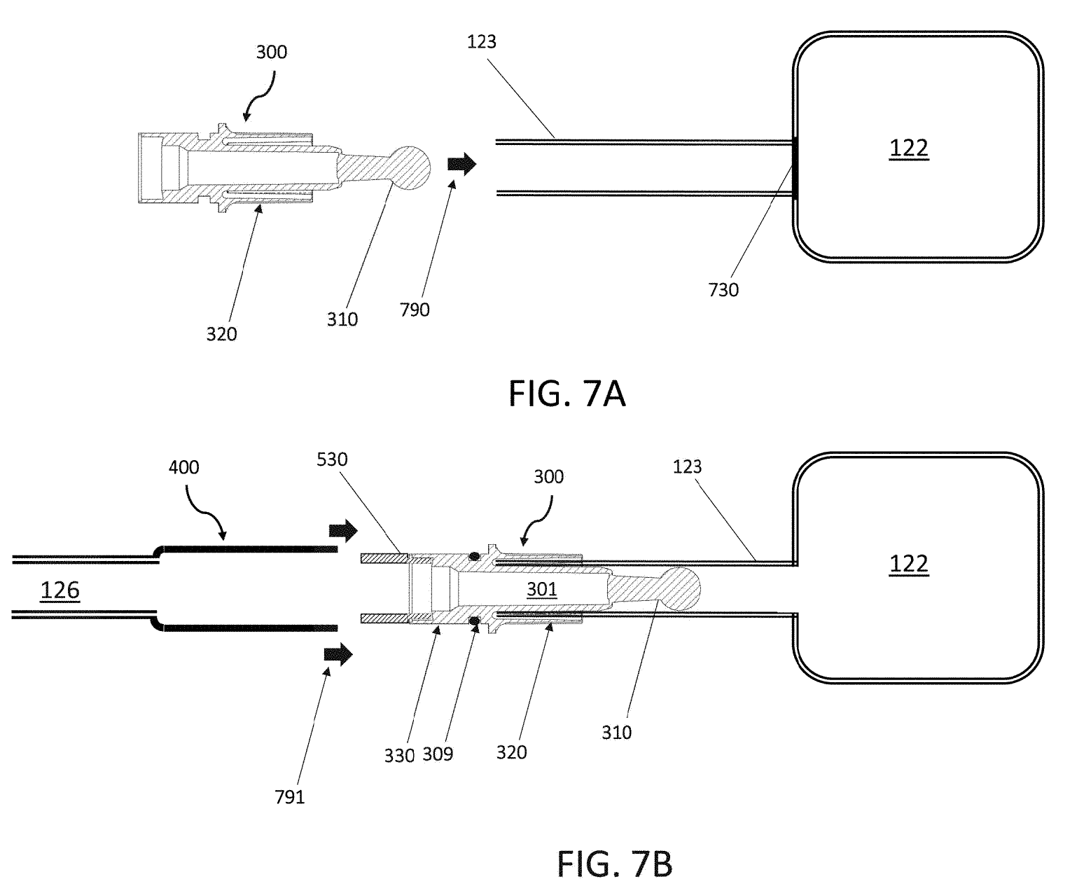

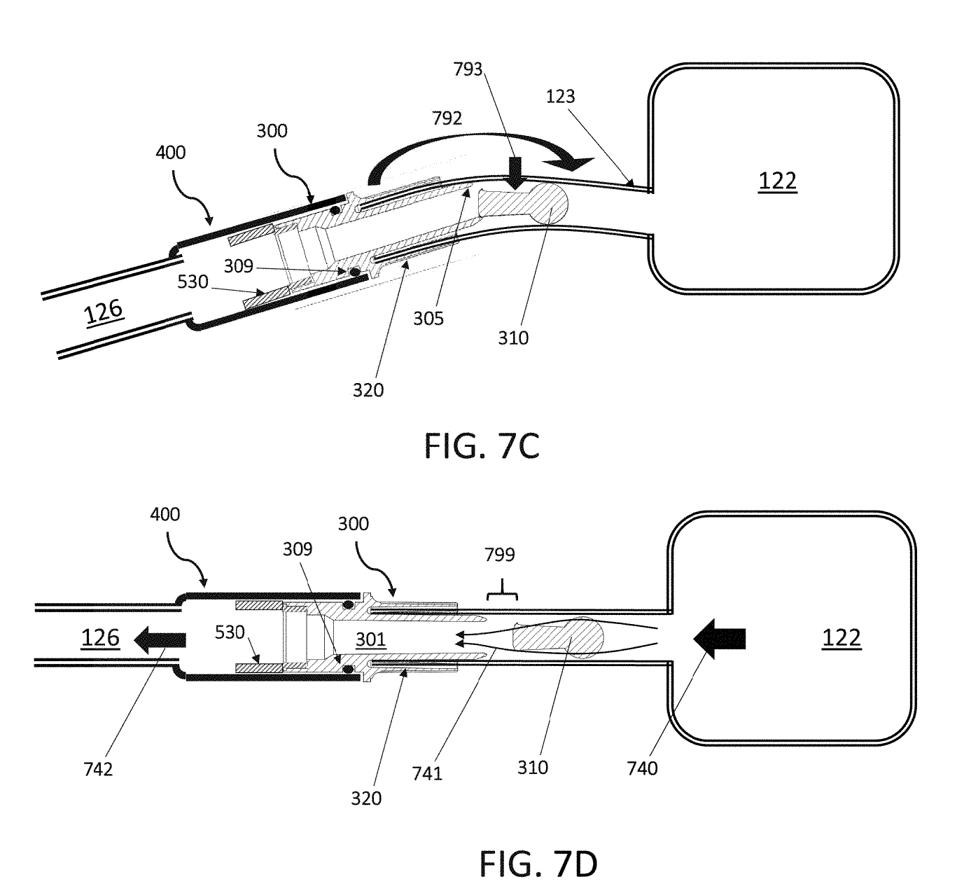

FIGS. 7A-7D are cross-section illustrations of the medical fluid line connector 300 being attached to the flexible medical fluid line 123 of the medical fluid bag 122 and subsequent attachment of a corresponding connector 400 and detachment of the breakaway body 310 from the medical fluid line connector 300. FIG. 7A shows an assembly step involving the breakaway body 310 of the medical fluid line connector 300 being inserted into the open end of the medical fluid line 122 and, subsequently, the open end of the medical fluid line 123 being secured to the medical fluid line connector 300 inside the peripheral sleeve 320. At this point, a medical fluid may already be contained in the medical fluid bag 122, and sealed by a breakable plug 730 that is breached after the medical fluid line connector 300 is sealed to the medical fluid line 123. Alternatively, the medical fluid can be added to the medical fluid bag 122 through the flexible medical fluid line 123 prior to the medical fluid line connector 300 sealing the open end of the flexible medical fluid tube 123, or the medial fluid bag 122 may contain another access port (not shown) configured to enable medical fluid to be added to the medical fluid bag 122 after the medical fluid line connector 300 is sealed to the flexible medical fluid line 123.

FIG. 7B shows the medical fluid line connector 300 including an interface component 530 attached to the open end 330 of the medical fluid line connector 300. The interface component 530 is configured to removeably and securely engage the corresponding connector 400 to the medical fluid line connector 300 using, for examples, exterior threads engaged with corresponding interior threads on the corresponding connector 400. The medical fluid line connector 300 also includes an O-ring seal 309 that interfaces with the corresponding connector 400 to ensure a leak-free fluid connection between the dialysis solution bag line 126 and the inner fluid cavity 301 of the medical fluid line connector 300. In operation, the corresponding connector 400 is placed over the open end 330 of the medical fluid line connector 300, as indicated by arrows 791, and then secured by the interface component 530 until sealingly engaged with the O-ring. Once connected, the corresponding connector 400 established a fluid communication between the dialysis solution bag lines 126 and the inner fluid cavity 301, with the breakaway body 310 prevent fluid communication between the barrier between the flexible medical fluid line 123 and the dialysis solution bag line 126.

FIG. 7C shows the corresponding connector 400 attached to the medical fluid line connector 300 and a force applied 729 to the medical fluid line connector 300 and the flexible medical fluid line 123 detaches the breakaway body 310 from the medical fluid line connector 300 at the breakaway region 305. The force applied 729 bends the flexible medical fluid line 123 and thereby applies a force 793 on the breakaway body 310 sufficient to sever or break the material of the breakaway region 305. Additionally, and as shown more clearly in FIG. 7D, the bending of the flexible medical fluid line 123 with the detached the breakaway body 310 serves to overcome the friction between the breakaway body 310 and the inner surface of the medical fluid line 123 and thereby translates the breakaway body 310 in the flexible medical fluid line 123 a small distance 799 away from the medical fluid line connector 300. FIG. 7D shows the force 792 removed from the flexible medical fluid line 123 and the breakaway body retained in the medical fluid line 123, with the small distance 799 being maintained by the interference fit between the breakaway body 310 and the flexible medical fluid line 123. As shown in FIG. 5, the tip region 307 of the breakaway body defines voids 319 that create fluid passageways around the breakaway body. Accordingly, FIG. 7D shows the flow direction of a medical fluid leaving 740 the medical fluid bag 122, passing around 741 the breakaway body, though the inner fluid cavity of the medical fluid connector 300 and into the attached dialysis solution bag line 126.

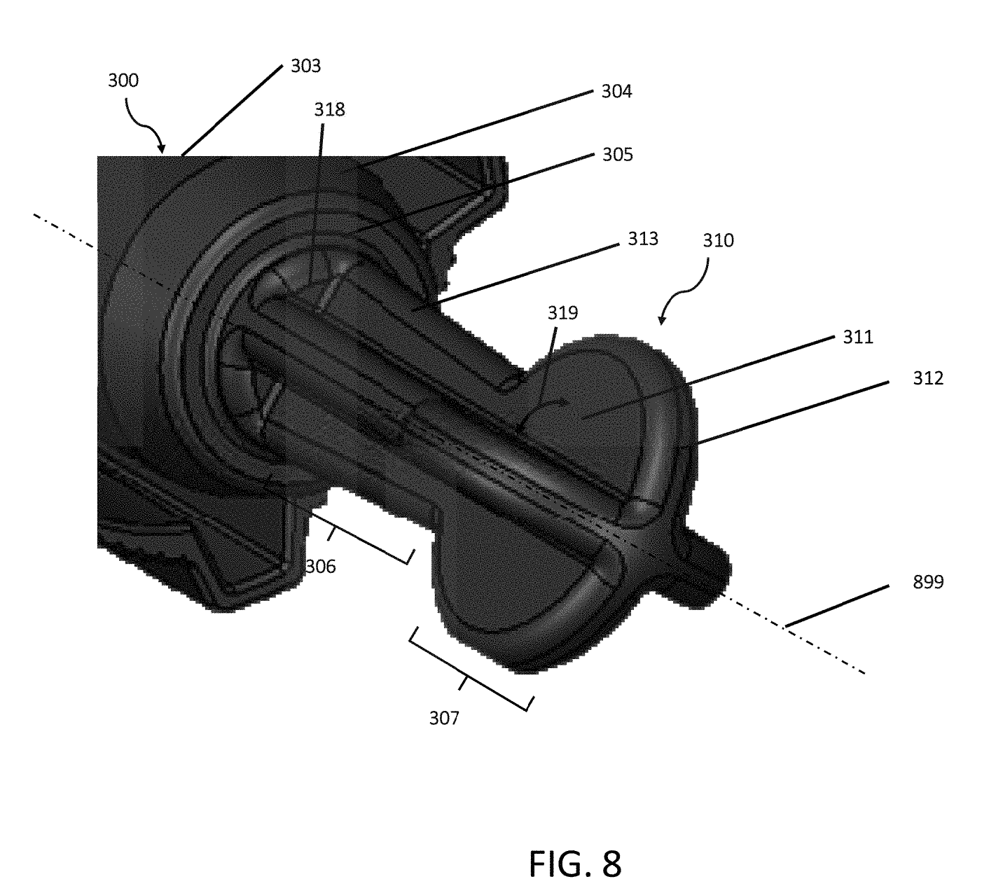

FIG. 8 is a perspective view of the breakaway body 310 attached to the main body 303 of the medical fluid line connector 300 of FIG. 5. FIG. 8 shows the details of the longitudinal ribs 311 of the breakaway body 310. The longitudinal ribs 311 define the voids 319 that span the length of the breakaway body 310. Each longitudinal rib 311 has a thickness and a longitudinal profile that defines a radial edge 312, 313. At the tip region 307 of the breakaway body 310, the radial edge 313 defines an arcuate shape and, together, the four longitudinal ribs form a generally spherical or ball-shaped feature at the tip region 307, with arcuate radial edges 313 sized and positioned to create an interface fit with the inner surface of the flexible medical fluid line 123 at the apexes of the arcuate radial edges 313. The size and shape of the arcuate radial edges 313 defines the degree of interaction between the breakaway body 310 and flexible medical fluid line 123. For example, if the arcuate radial edges 313 form an overall larger effective diameter of the tip region 307, then a stronger interference fit may be created with the same flexible medical fluid line 123. Additionally, the thickness of each longitudinal rib 311 (or, more generally, the cross-sectional profile on the plane normal to the longitudinal axis 899 of the breakaway body) defines the size of the voids 319 between adjacent longitudinal ribs 311, and the voids 319 in the tip region 307 form the fluid passageways. In FIG. 8, the longitudinal ribs 311 define a constant core thickness of the breakaway body 1310, and, because the breakaway region 305 is circular, the voids 319 between the longitudinal ribs 311 end at terminal region 318 defined as the surface where the voids 319 end to meet the circular breakaway region 305 at the main body 302.

FIGS. 9A and 9B are cross-sectional and exploded views of interface components 530 attached to the open end 330 of the medical fluid line connector of FIG. 5. FIG. 9A shows the medical fluid line connector 300 including the breakaway body, O-ring 309, and an open end 330. An interface component 530 is attached to the open end 330 of the fluid line connector 300 and includes exterior threads 531 configured to interface with corresponding threads on the corresponding connector 400. Also, a trifurcated silicone disk 350 is disposed in the open end 330 of the fluid line connector 300 and held in place by the interference component 530. In operation, the trifurcated silicone disk 350 prevents the medical fluid line connector 300 from forming an open fluid passageway when the breakaway body 310 is connected by requiring negative pressure (such as from the pumps of the PF cycler 102) to drive fluid across trifurcated silicone disk 350. FIG. 9B shows the components of FIG. 9A in isolation.

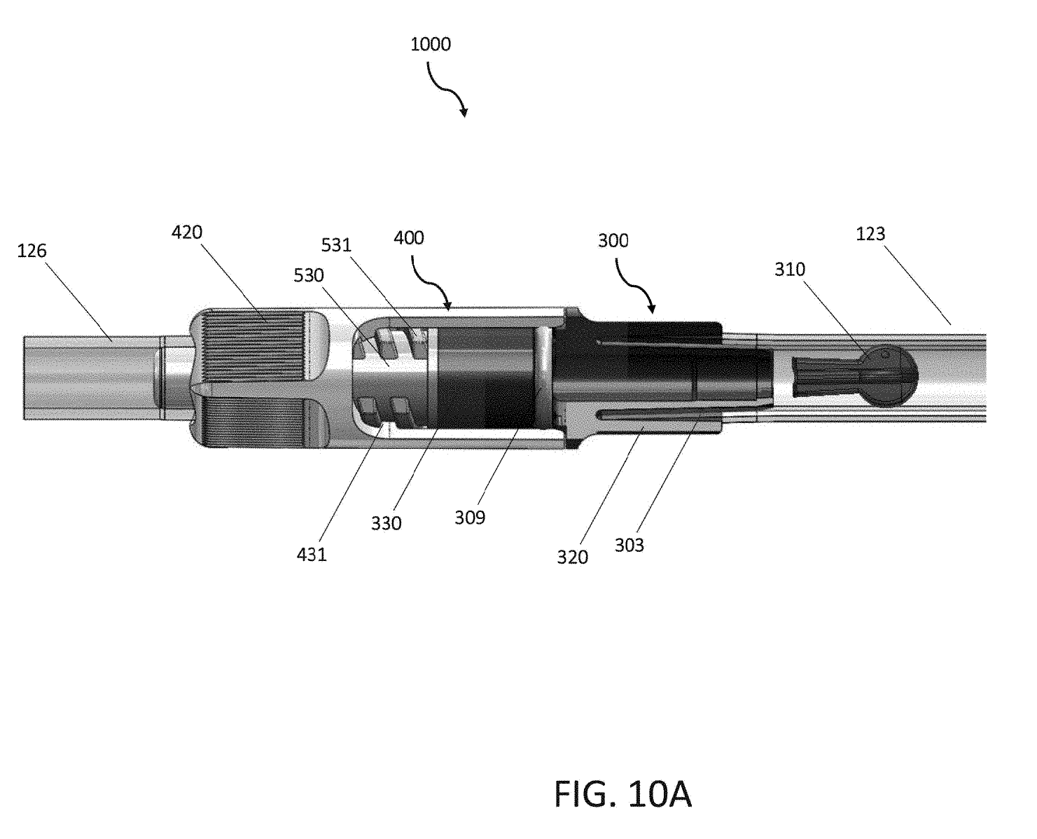

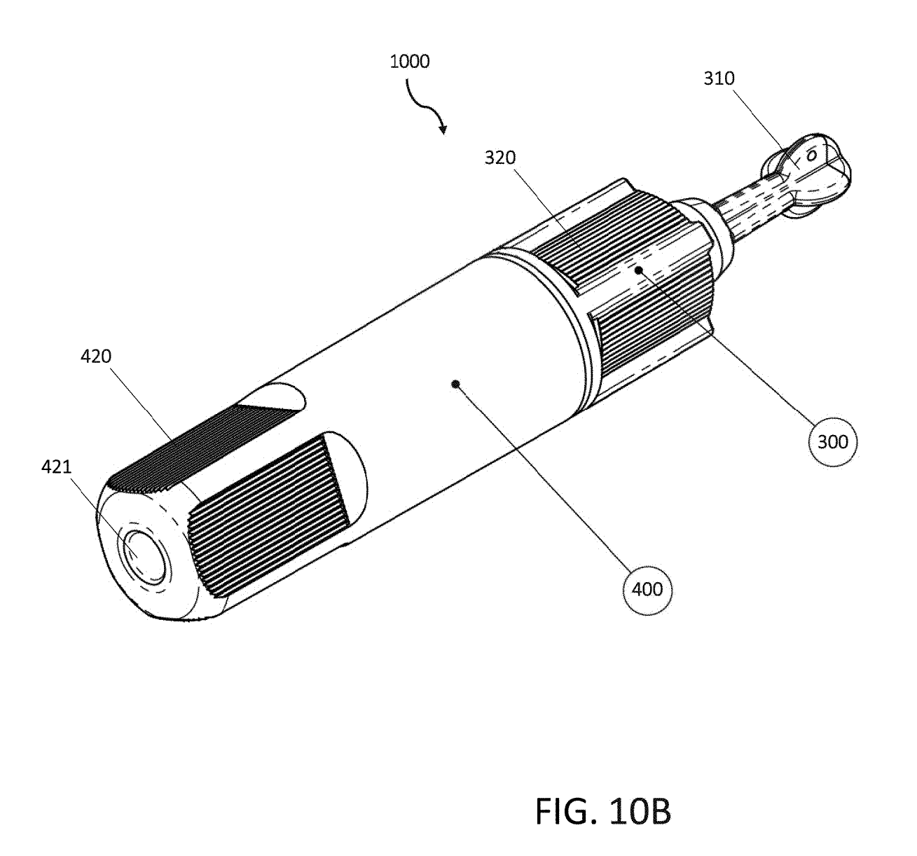

FIGS. 10A and 10B are cross-sectional and perspective illustrations, respectively, of a corresponding connector 400 coupled to the interface components of the medical fluid line connector 300 of FIGS. 9A and 9B. FIG. 10A illustrates the operating assembly 1000 of a corresponding connector 400 connecting a dialysis solution bag line 126 to a medical fluid line 123 via coupling with the medical fluid line connector 300. The corresponding connector 400 includes interior threads 431 in threaded engagement with the exterior threads 531 of the interface component 530 attached to the open end 330 of the medical fluid line connector 300. The corresponding connector 400, in this coupled configuration, extends over the open end 330 of the medical fluid line connector 300 and engages the O-ring 309 to further seal the connection between the dialysis solution bag line 126 and the inner fluid cavity 301 of the medical fluid line connector 300. As shown, the breakaway body 310 of the medical fluid line connector 300 is detached, indicating that medical fluid line connector's 300 seal on the medical fluid line 123 is breached and the corresponding connector's 400 coupling to the medical fluid line connector 300 has established a fluid passageway between the dialysis solution bag line 126 and the medical fluid line 123. The exterior of the corresponding connector 400 includes a gripping region 420 enabling a user to, for example grasp the peripheral sleeve 320 of the medial fluid line connector 300 and the gripping region 420 in order to apply a torque across the assembly to engage or disagreed the threaded engagement between the corresponding connector 400 and the interface component 530. Also, the corresponding connector 400 includes an opening 421 configured to receive the dialysis solution bag line 126.

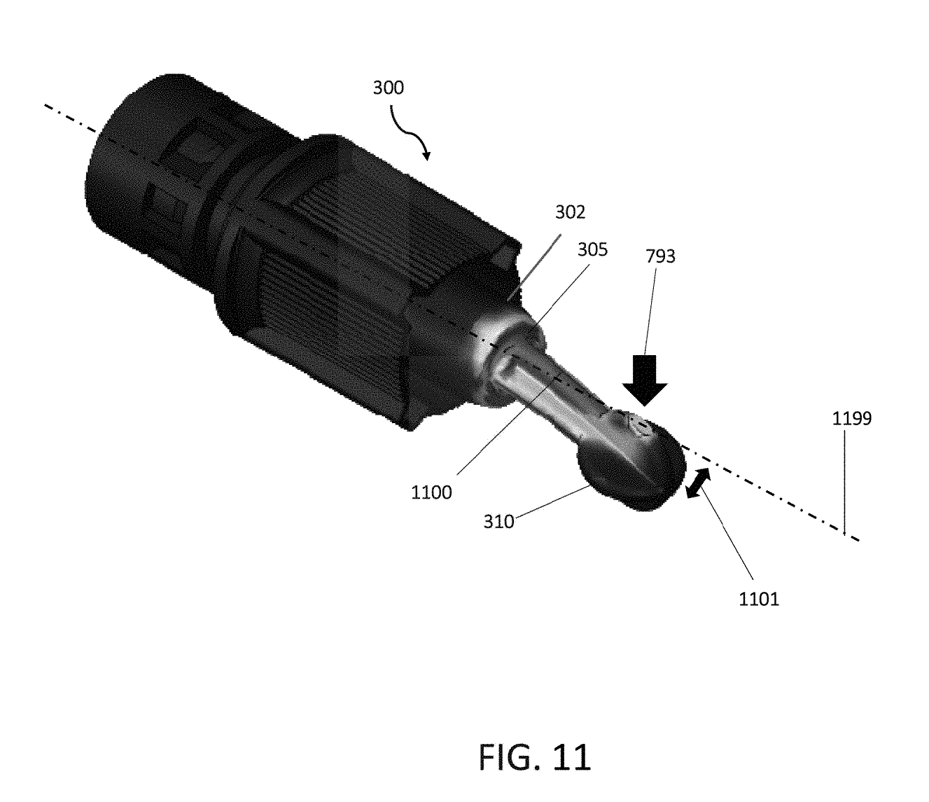

FIG. 11 is a perspective illustration of a computational simulation of the deflection of the breakaway body 310 of the medical fluid line connector 300 of FIG. 5. FIG. 11 shows a deflection 1101 of the breakaway body 310 with respect to the longitudinal axis 1199 of the medical fluid line connector 300 in response to an applied force 793. The shading 1100 illustrates the applied force 793 causes tensile stress in the breakaway region 305 sufficient to cause separation of the breakaway body from the main body 302.

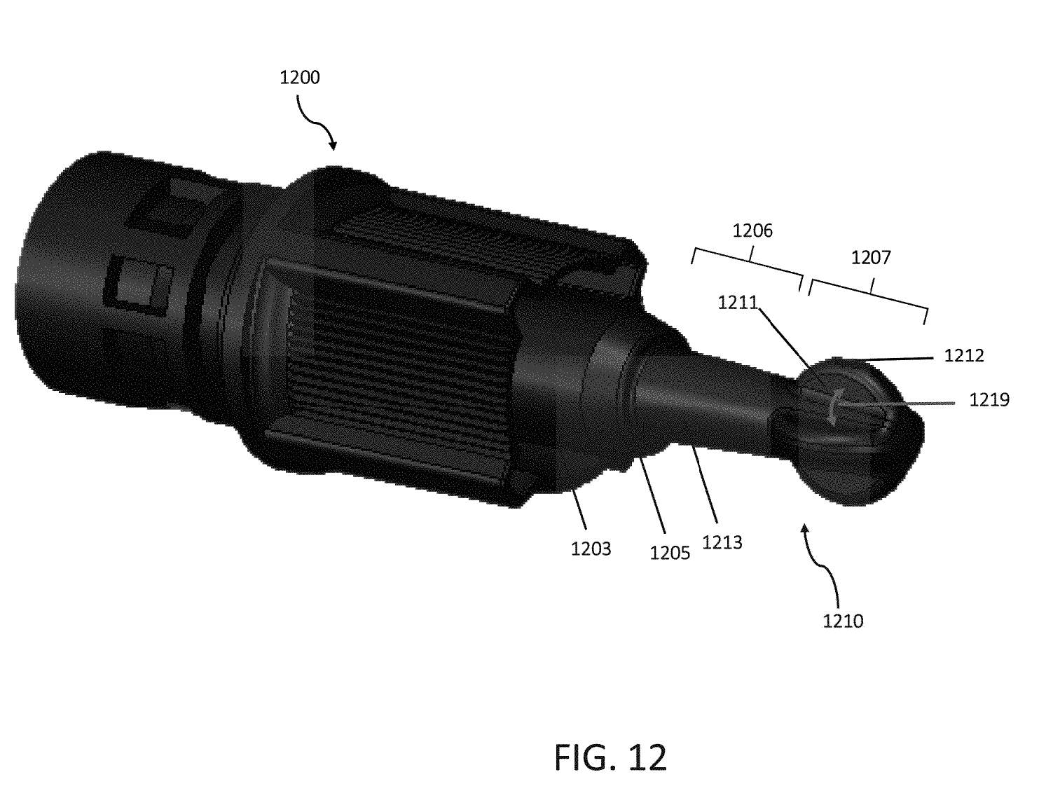

FIG. 12 is a perspective illustration of an alternative design of a medical fluid line connector 1200. The medical fluid line connector 1200 includes a main body 1203 and a breakaway body 1210 joined to the main body 1203 at a breakaway region 1205. The breakaway body 1210 includes longitudinal ribs 1211 defining in a tip region 1207 and an adjacent base region 1206 having a simple conical exterior 1213. The longitudinal ribs 1211 define arcuate radial edges 1212 sized and dimensioned to create a friction interference fit with the inner surface of a flexible medical fluid line 123. The conical exterior 1213 extends through the tip region 1207 between the longitudinal ribs 1211. In operation, the breakaway body 1210, when detached from the main body 1203 in a flexible medical fluid line 123, forms fluid passageways through the voids 1219 between the longitudinal ribs 1211, and the completely around the conical exterior 1213 of the base region 1206.

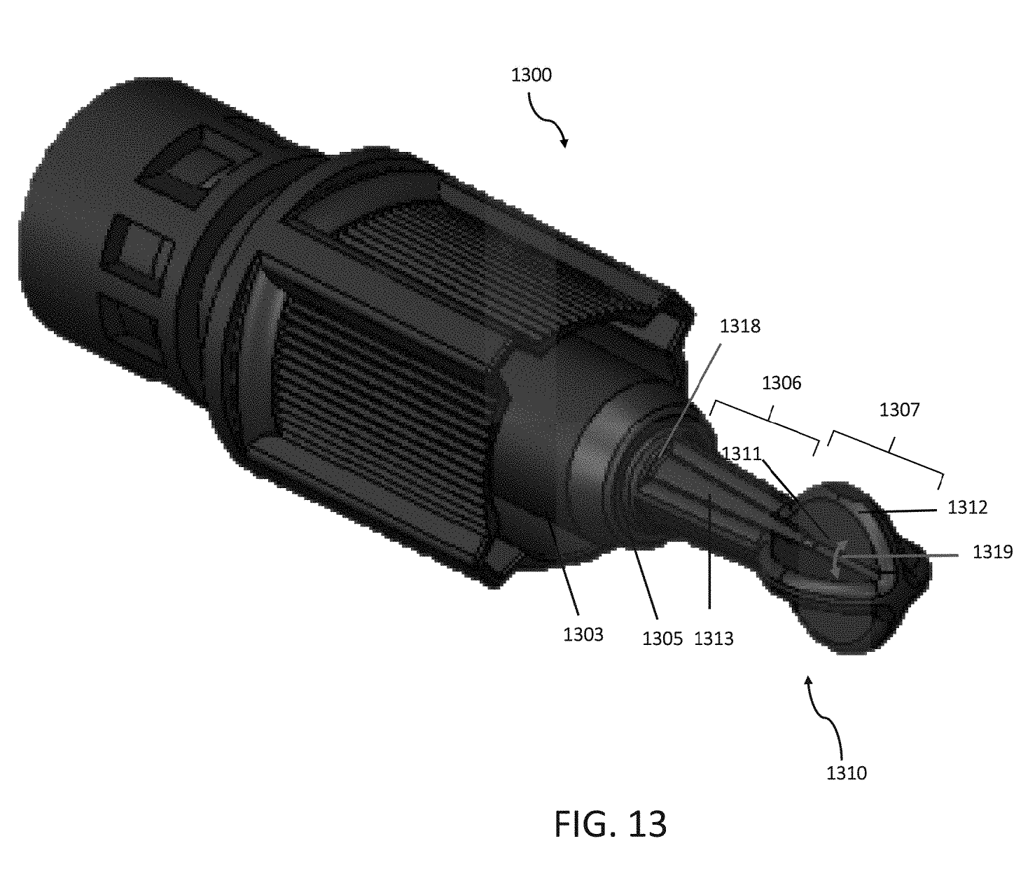

FIG. 13 is a perspective illustration of an alternative design of a medical fluid line connector. The medical fluid line connector 1300 includes a main body 1303 and a breakaway body 1310 joined to the main body 1303 at a breakaway region 1205. The breakaway body 1310 includes conical surface sections 1313 in a base region 1306 and longitudinal ribs 1311 that span the length of the breakaway body 1310. The longitudinal ribs 1311 in a tip region 1307 of the breakaway body 1310 define arcuate radial edges 1312 sized and dimensioned to create a friction interference fit with the inner surface of a flexible medical fluid line 123. The conical surface sections 1313 in the base region extend through the tip region 1307 between the longitudinal ribs 1311 and create an increasing core thickness of the breakaway body 1310 (i.e., the diameter at the root of the longitudinal ribs 1311) from the tip region 1307 to the breakaway region 1305. In operation, the breakaway body 1310, when detached from the main body 1303 in a flexible medical fluid line 123, forms fluid passageways through the voids 1319 between the longitudinal ribs 1311. The increasing core thickness of the breakaway body 1310 reduces the size of the terminal region 1318 of the voids 1319 and outwardly defects fluid flow between the longitudinal ribs 1311 across the base region 1306, which can reduce the turbulence generated by a fluid flow though the voids 1319.

While the medical fluid line connectors have been described as parts of PD systems, they can be used in any of various other types of medical fluid pumping systems, including hemodialysis systems, blood perfusion systems, and intravenous infusion systems.

Similarly, while the medical fluid bags have been described as containing dialysis solution, they can alternatively or additional contain any of various other types of medical fluids, including blood, saline, drugs, etc.

Referring now to FIG. 3, in some instances, the medical fluid bag 122 contain any fluid solution for use in a medical procedure. While the flexible medical fluid line has been shown to connect to a medical fluid bag 122, in some instances the flexible medical fluid line is a fluid supply line.

Referring now to FIG. 5, in some instances the medical fluid line connector 300 in constructed from a polymer, and injection molded. In some instances, the peripheral sleeve 320 is not used in securing the flexible medical fluid line 123 to the main body 123. In some instances, the tip region 307 defines an elliptical shape or otherwise includes a rounded profile free of sharp angles to avoid damage to the inner surface of the flexible medical fluid tube. In some instances, the breakaway body 310 defines inner fluid passages configured to replace or supplement the fluid around the breakaway body 310 by permitting a flow of fluid though a portion of the breakaway body 310. While the breakaway body has been shown as symmetric, in some instances the breakaway body 310 is nonsymmetrical and may include a feature configured to be pressed on by a user to facilitate the application of the breakage force onto to breakaway body 310. While the medical fluid line connector has been shown to have a tubular main body 310, in some instances the medical fluid line connector has a square or other non-circular exterior or interior cross section. While the breakaway body 310 has been shown as includes a base region and a tip region, in some instances the breakaway body includes only a tip region that connects to the breakaway region 305 of the main body 302.

Referring now to FIGS. 6A and 6B, while the breakaway region 305 has been shown to be a thin-walled or frangible region 305, in some instances, the breakaway region includes a different material from the main body 302. In some instances, the breakaway region 302 includes a plurality of indentations to reduce the breakaway force.

Referring now to FIGS. 9A and 9B, in some instances, the interface component 530 is integrated into the medical fluid line connector.

Referring now to FIGS. 10A and 10B, in some instances, the connection between the corresponding connector 400 and the interface component 530 is a Luer lock or similar.

A number of embodiments of the invention have been described. Nevertheless, it will be understood that various modifications may be made without departing from the spirit and scope of the invention. Accordingly, other embodiments are within the scope of the following claims.

What is claimed is:

1. A medical fluid line connector comprising:
a main body configured to accept a corresponding connector, the main body defining a first opening to permit fluid communication between an interior region of the medical fluid line connector and the corresponding connector when the corresponding connector is connected to the main body; and
a breakaway body detachably connected to the main body, the breakaway body being configured to be inserted into an open end of a flexible medical fluid line, the breakaway body having a portion configured to create an interference fit against an inner surface of the flexible medical fluid line when the breakaway body is inserted into the flexible medical fluid line, and the breakaway body and the main body being configured such that detachment of the breakaway body from the main body forms a second opening in the main body to permit fluid communication between the medical fluid line and the interior region of the medical fluid line connector.

2. The medical fluid line connector of claim 1, wherein the breakaway body at least partially defines a fluid passageway configured to permit fluid flowing through the medical fluid line to flow past the breakaway body and into the second opening.

3. The medical fluid line connector of claim 1, wherein an intersection of the main body and the breakaway body define a breakaway region configured to enable a force applied to the breakaway body to separate the breakaway body from the main body at the breakaway region.

4. The medical fluid line connector of claim 3, wherein the breakaway region comprises a thin wall region.

5. The medical fluid line connector of claim 3, wherein the breakaway region comprises a frangible region.

6. The medical fluid line connector of claim 1, further comprising:
an exterior sleeve around a periphery of the main body, a portion of the exterior sleeve radially spaced from the main body to define a sleeve cavity, the sleeve cavity being configured to receive the open end of the medical fluid line and seal the open end of medical fluid line around an exterior surface of the main body.

7. The medical fluid line connector of claim 1, wherein the breakaway body comprises a plurality of protrusions, and a portion of a terminal edge of each protrusion is sized and positioned to create the interference fit against the inner surface of the flexible tube.

8. The medical fluid line connector of claim 7, wherein the terminal edges of the plurality of protrusions define an arcuate shape.

9. The medical fluid line connector of claim 8, wherein the plurality of protrusions together form a substantially spherical shape.

10. The medical fluid line connector of claim 7, wherein the plurality of protrusions define a corresponding plurality of gaps between adjacent protrusions such that the protrusions and the inner surface of the flexible tube together define a plurality of fluid passageways configured to permit fluid flowing through the medical fluid line to flow past the breakaway body and into the second opening.

11. The medical fluid line connector of claim 7, wherein the protrusions comprise longitudinal ribs.

12. The medical fluid line connector of claim 11, wherein the longitudinal ribs span the length of the breakaway body.

13. The medical fluid line connector of claim 1, further including a connector interface body attached to the main body, the connector interface body comprising interface features configured to mate with corresponding interface features on the corresponding connector and securely attach the corresponding connector to the medical fluid line connector.

14. The medical fluid line connector of claim 1, wherein the flexible medical fluid line is coupled to a container containing fluid for use in a dialysis operation, and the connector is configured to seal the bag by sealing the open end of the flexible medical fluid line.

15. The medical fluid line connector of claim 1, wherein the corresponding connector is a corresponding connector of a medical fluid line of a dialysis system.

16. The medical fluid line connector of claim 1, wherein the medical fluid line extends from a medical fluid container.

17. The medical fluid line connector of claim 16, wherein the medical fluid container is a dialysis solution container.

18. The medical fluid line connector of claim 1, wherein the medical fluid line extends from a medical fluid bag.

19. The medical fluid line connector of claim 18, wherein the medical fluid bag is a dialysis solution bag.

20. A system comprising:
a medical fluid container,
a flexible medical fluid line connected to the medical fluid container; and
a medical fluid line connector connected to the medical fluid line, the medical fluid line connector comprising
a main body configured to accept a corresponding connector, the main body defining a first opening to permit fluid communication between an interior region of the medical fluid line connector and the corresponding connector when the corresponding connector is connected to the main body; and
a breakaway body detachably connected to the main body, the breakaway body disposed inside of an open end of the flexible medical fluid line, the breakaway body having a portion defining an interference fit against an inner surface of the flexible medical fluid line, and the breakaway body and the main body being configured such that detachment of the breakaway body from the main body forms a second opening in the main body to permit fluid communication between the medical fluid container and the interior region of the medical fluid line connector.

21. A method of creating fluid communication between a medical fluid container and a medical fluid line connector that is secured to a medical fluid line, the medical fluid line being coupled to the medical fluid container, the method comprising:
separating a breakaway body of the medical fluid line connector from a main body of the medical fluid line connector in a manner to create a gap between the breakaway body and the main body,
wherein a portion of the breakaway body forms an interference fit with an inner surface of the medical fluid line to maintain the gap between the breakaway body and the main body.

22. The method of claim 21, wherein separating the breakaway body from the main body comprises bending the breakaway body relative to the main body.

23. The method of claim 21, wherein separating the breakaway body from the main body comprises rotating the breakaway body relative to the main body.

24. The method of claim 21, further comprising connecting tubing to an end region of the medical fluid line connector opposite an end region of the medical fluid line connector to which the medical fluid line is connected.

25. The method of claim 21, further comprising passing medical fluid from the medical fluid container to the medical fluid line connector via the medical fluid line.

26. The method of claim 21, wherein the medical fluid container is a medical fluid bag.

27. The method of claim 26, wherein the medical fluid bag is a dialysis solution bag.

28. The method of claim 21, wherein the medical fluid container is a dialysis solution container.

29. The method of claim 21, wherein at least a portion of the breakaway body forms a fluid passageway to permit a fluid flow in the medical fluid line to pass the breakaway body and into the medical fluid line connector.
